(19) United States
(12) Patent Application Publication
(10) Pub. No.: US 2020/0205711 A1
(43) Pub. Date: Jul. 2, 2020

(54) PREDICTING DEPRESSION FROM NEUROELECTRIC DATA

(71) Applicant: X Development LLC, Mountain View, CA (US)

(72) Inventors: Sarah Ann Laszlo, Mountain View, CA (US); Georgios Evangelopoulos, Venice, CA (US); Pramod Gupta, Mountain View, CA (US)

(21) Appl. No.: 16/284,556

(22) Filed: Feb. 25, 2019

(30) Foreign Application Priority Data
Dec. 28, 2018 (GR) 20180100572

(51) Int. Cl. A61B 5/16; A61B 5/0484; A61B 5/0482; A61B 5/00; G06K 9/00

(52) U.S. Cl. CPC A61B 5/165; A61B 5/04842; A61B 5/0482; G06K 9/00536; A61B 5/7275; A61B 5/7267; A61B 5/7264

(57) ABSTRACT

Methods, systems, and apparatus, including computer programs encoded on a computer storage medium, for causing a stimulus presentation system to present first content to a patient. Obtaining, from a brainwave sensor, electroencephalography (EEG) signals of the patient while the first content is being presented to the patient. Identifying, from within the EEG signals of the patient, first brainwave signals associated with a first brain system of the patient, the first brainwave signals representing a response by the patient to the first content. Determining, based on providing the first brainwave signals as input features to a machine learning model, a likelihood that the patient will experience a type of depression within a period of time. Providing, for display on a user computing device, data indicating the likelihood that the patient will experience the type of depression within the period of time.

### CROSS-REFERENCE TO RELATED APPLICATION

This application claims priority to Greek Patent Application No. 20180100572, filed on Dec. 28, 2018, entitled “PREDICTING DEPRESSION FROM NEUROELECTRIC DATA,” the entirety of which is hereby incorporated by reference.

### TECHNICAL FIELD

This disclosure generally relates to brainwave measurements. More particularly the disclosure relates to processes for using brainwave measurements to predict the likelihood that a patient will experience depression in the future.

### BACKGROUND

Depression is a problem for many people. Early detection of a patient's likelihood to experience depression can permit doctors to provide necessary treatment before serious symptoms occur. Brain activity can serve as an early indicator of a patient's future risk of depression.

### SUMMARY

In general, the disclosure relates to a machine learning system that predicts future changes in the mental health of a patient based on neuroelectric signals of the patient. The system can provide a binary output or probabilistic output indicating the likelihood that a patient will experience depression over a period of time in the future. More specifically, the system processes a current sample of electroencephalogram (EEG) signals for a patient and predicts the likelihood that the patient will become depressed over a predefined time period (e.g., several months or several years). The system can correlate EEG signals from specific brain systems (e.g., the reward system, emotion system, and/or resting state) to predict future changes in mental health.

In general, innovative aspects of the subject matter described in this specification can be embodied in methods that include the actions of causing a stimulus presentation system to present first content to a patient. Obtaining, from a brainwave sensor, electroencephalography (EEG) signals of the patient while the first content is being presented to the patient. Identifying, from within the EEG signals of the patient, first brainwave signals associated with a first brain system of the patient, the first brainwave signals representing a response by the patient to the first content. Determining, based on providing the first brainwave signals as input features to a machine learning model, a likelihood that the patient will experience a type of depression within a period of time. Providing, for display on a user computing device, data indicating the likelihood that the patient will experience the type of depression within the period of time. Other implementations of this aspect include corresponding systems, apparatus, and computer programs, configured to perform the actions of the methods, encoded on computer storage devices.

These and other implementations can each optionally include one or more of the following features.

In some implementations, the first content is selected to trigger a response by a particular brain system of the patient.

In some implementations, the first brain system is a dopaminergic system or amygdala emotional system.

Some implementations include causing the stimulus presentation system to present second content to the patient, the second content being different from the first content. Obtaining EEG signals of the patient while the second content is being presented to the patient. Identifying, from within the EEG signals of the patient, second brainwave signals associated with a second brain system of the patient, the second brainwave signals representing a response by the patient to the second content, where determining the likelihood that the patient will experience the type of depression within the period of time includes determining, based on providing the first brainwave signals and the second brainwave signals as input features to the machine learning model, the likelihood that the patient will experience the type of depression within the period of time.

In some implementations, the first brain system is a reward system and the second brain system is an emotion system.

Some implementations include obtaining EEG signals of the patient while no content is presented to the patient, and identifying, from within the EEG signals of the patient, third brainwave signals associated with a resting state of the patient, where determining the likelihood that the patient will experience the type of depression within the period of time includes determining the likelihood that the patient will experience the type of depression within the period of time based on the first brainwave signals, the second brainwave signals, and the third brainwave signals.

In some implementations, determining the likelihood that the patient will experience the type of depression within the period of time includes determining a severity of the type of depression.

In some implementations, the machine learning model is a convolutional neural network.

In some implementations, the machine learning model is a supervised machine learning model configured to be adaptive to actual patient diagnoses of depression.

In some implementations, the machine learning model is trained based on more than one hundred data sets of clinical test data.

In some implementations, the type of depression includes major depressive disorder or post-partum depression.

In some implementations, the first content includes interactive content configured to test the patient's responses to receiving rewards and taking risks.

In some implementations, the first content includes a sequence of images representing positive, neutral, and negative emotional stimuli.

In general, innovative aspects of the subject matter described in this specification can be embodied in methods that include the actions of causing a stimulus presentation system to present first content to a patient, where the first content includes interactive content configured to test the patient's responses to receiving rewards and taking risks, Obtaining, from a brainwave sensor, electroencephalography (EEG) signals of the patient while the first content is being presented to the patient. Identifying, from within the EEG signals of the patient, first brainwave signals associated with a dopaminergic brain system of the patient, where the first brainwave signals represent a response by the patient to the first content. Causing the stimulus presentation system to present second content to the patient, the second content being different from the first content, the second content including a sequence of images representing positive, neutral, and negative emotional stimuli. Obtaining EEG signals of the patient while the second content is being presented to the patient. Identifying, from within the EEG signals of the patient, second brainwave signals associated with the amygdala and greater emotion processing system of the patient, where the second brainwave signals represent a response by the patient to the second content. Determining, based on providing the first brainwave signals and the second brainwave signals as input features to a machine learning model, a likelihood that the patient will experience a type of depression within a period of time. Providing, for display on a user computing device, data indicating the likelihood that the patient will experience the type of depression within the period of time. Other implementations of this aspect include corresponding systems, apparatus, and computer programs, configured to perform the actions of the methods, encoded on computer storage devices.

These and other implementations can each optionally include one or more of the following features.

In some implementations, determining the likelihood that the patient will experience the type of depression within the period of time includes determining a severity of the type of depression.

In some implementations, the machine learning model is a convolutional neural network.

In some implementations, the machine learning model is a supervised machine learning model configured to be adaptive to actual patient diagnoses of depression.

In some implementations, the machine learning model is trained based on more than one hundred data sets of clinical test data.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts block diagram of an example neuroelectric depression prediction system in accordance with implementations of the present disclosure.

FIG. 2 depicts an example brainwave sensor system and stimulus presentation system according to implementations of the present disclosure.

FIG. 3 depicts a flowchart of an example process for using neuroelectric data to predict a patient's likelihood of experiencing depression in the future in accordance with implementations of the present disclosure.

FIG. 4 depicts a schematic diagram of a computer system that may be applied to any of the computer-implemented methods and other techniques described herein.

Like reference numbers and designations in the various drawings indicate like elements.

### DETAILED DESCRIPTION

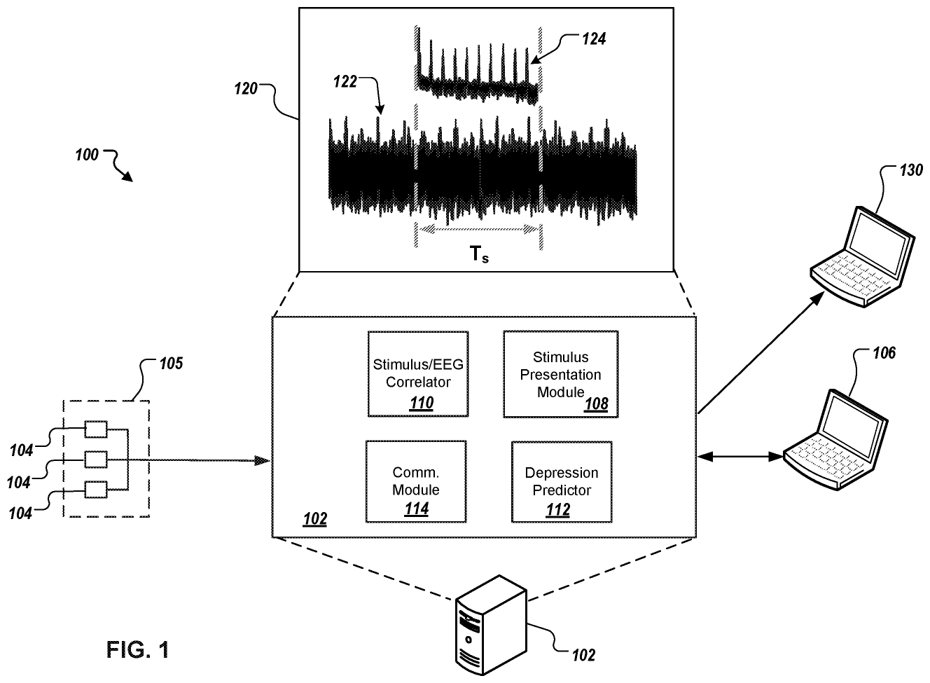

FIG. 1 depicts a block diagram of an example neuroelectric depression prediction system 100. The system includes a depression prediction module 102 which is in communication with brainwave sensors 104, a stimulus presentation system 106, and, optionally, one or more user computing devices 130. The depression prediction module 102 can be implemented in hardware or software. For example, the depression prediction module 102 can be a hardware or a software module that is incorporated into a computing system such as a server system (e.g., a cloud-based server system), a desktop or laptop computer, or a mobile device (e.g., a tablet computer or smartphone). The depression prediction module 102 includes several sub-modules which are described in more detail below. As a whole, the depression prediction module 102 receives a patient's brainwave signals (e.g., EEG signals) from the brainwave sensors 104 while stimuli are presented to a patient. The depression prediction module 102 identifies brainwaves from particular brain systems that are generally responsive to specific media content presented as stimuli. The depression prediction module 102 uses a machine learning model to analyze identified brainwaves and predict the likelihood that the patient will experience depression within a predefined time in the future.

For example, the depression prediction module 102 obtains EEG data of a patient's brainwaves while the patient is presented with stimuli that are configured to trigger responses in brain systems related to depression. As described in more detail below, the stimuli can include content designed to trigger responses in brain systems such as the dopaminergic reward system in general and the amygdala in specific. The depression prediction module 102 can correlate the timing of the content presentation with the brainwaves in both the temporal and spatial domains to identify brainwaves or patterns of brainwaves associated with the applicable brain system. The depression prediction module 102 analyzes the brainwave signals from one or more brain systems to identify stimulus response patterns that are indicative of a future risk of depression. As discussed below, the depression prediction module 102 can employ a machine learning model trained on hundreds of clinical test data sets to predict a patient's future likelihood of experiencing depression. The depression prediction module 102 can provide a binary output or probabilistic output (e.g., a risk score) indicating the likelihood that the patient's will experience depression over a predefined period of time. For example, the depression prediction module 102 can predict the likelihood that the patient will become depressed within several months (e.g., 6 months, 9 months, 12 months, or 18 months) from the time that the patient's brainwaves are measured and analyzed. In some implementations, the depression prediction module 102 can predict how severe the depression is likely to be (e.g., mild, moderate, or severe). For example, the depression prediction module 102 can predict the likely severity at each of those example time points, in addition to the binary depressed/non-depressed classification. The depression prediction module 102 sends the output data to a computing device 130 associated with the patient's doctor (e.g., a psychiatrist) such as the doctor's office computer or mobile device.

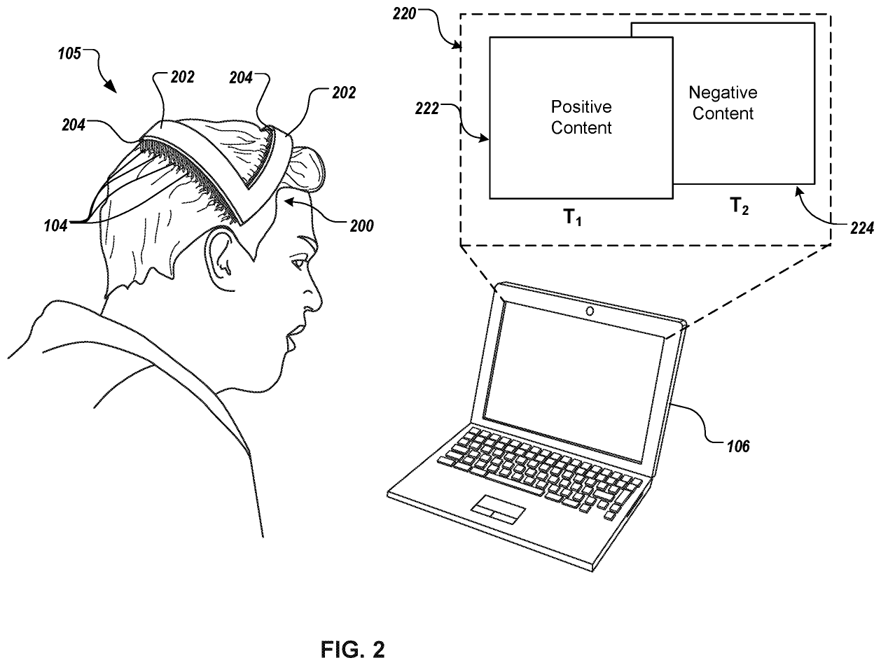

In general, any sensors capable of detecting brainwaves may be used. For example, the brainwave sensors 104 can be one or more individual electrodes (e.g., multiple EEG electrodes) that are connected to the depression prediction module 102 by wired connection. The brainwave sensors 104 can be part of a brainwave sensor system 105 that is in communication with the depression prediction module 102. A brainwave sensor system 105 can include multiple individual brainwave sensors 104 and computer hardware (e.g., processors and memory) to receive, process, and/or display data received from the brainwave sensors 104. Example brainwave sensor systems 105 can include, but are not limited to, EEG systems, a wearable brainwave detection device (e.g., as described below in reference to FIG. 2 below), a magnetoencephalography (MEG) system, and an Event-Related Optical Signal (EROS) system, sometimes also referred to as “Fast NIRS” (Near Infrared spectroscopy). A brainwave sensor system 105 can transmit brainwave data to the depression prediction module 102 through a wired or wireless connection.

FIG. 2 depicts an example brainwave sensor system 105 and stimulus presentation system 106. The sensor system 105 is a wearable device 200 which includes a pair of bands 202 that fit over a user's head. Specifically, the wearable device 200 includes one band which fits over the front of a user's head and the other band 202 which fits over the back of a user's head, securing the device 200 sufficiently to the user during operation. The bands 202 include a plurality of brainwave sensors 104. The sensors 104 can be, for example, electrodes configured to sense the user's brainwaves through the skin. For example, the electrodes can be non-invasive and configured to contact the user's scalp and sense the user's brainwaves through the scalp. In some implementations, the electrodes can be secured to the user's scalp by an adhesive.

The sensors 104 are distributed across the rear side 204 of each band 202. In some examples, the sensors 104 can be distributed across the bands 202 to form a comb-like structure. For example, the sensors 104 can be narrow pins distributed across the bands 202 such that a user can slide the bands 202 over their head allowing the sensors 104 to slide through the user's hair, like a comb, and contact the user's scalp. Furthermore, the comb-like structure sensors 104 distributed on the bands 202 may enable the device 200 to be retained in place on the user's head by the user's hair. In some implementations, the sensors 104 are retractable. For example, the sensors 104 can be retracted into the body of the bands 202.

In some examples, the sensors 104 are active sensors. For example, active sensors 104 are configured with amplification circuitry to amplify the EEG signals at the sensor head prior to transmitting the signals to a receiver in the depression prediction system 100 or the stimulus presentation system 105.

The stimulus presentation system 106 is configured to present content 220 to the patient while the patient's brainwaves are measured. For example, the stimulus presentation system 106 can be a multimedia device, such as a desktop computer, a laptop computer, a tablet computer, or another multimedia device. The content 220 is designed or selected to trigger responses in particular brain systems that are predictive of depression. For example, the content 220 can be selected to trigger responses in a patient's reward system (e.g., the dopaminergic system) or emotion system (e.g., the amygdala).

The content 220 can include, but is not limited to, visual content such as images or video, audio content, or interactive content such as a game. For example, emotional content can be selected to measure the brain's response to the presentation of emotional stimuli. Emotional content can include the presentation of a series of positive images 222 (e.g., a happy puppy), negative images 224 (e.g., a dirty bathroom), and neutral images (e.g., a stapler). The emotional images can be presented randomly or in a pre-selected sequence. As another example, risk/reward content can be used to measure the brain's response to receiving a reward. Risk/reward content can include, but is not limited to, an interactive game where the patient choose one of two doors and can either win or lose a small amount of money (e.g., win=$1.00, lose=$0.50) depending on which door they choose. The order of wins and losses can be random. In some implementations, no content is presented, e.g., in order to measure the brain's resting state to obtain resting state brainwaves.

In some implementations, the wearable device 200 is in communication with the stimulus presentation system 106, e.g., a laptop, tablet computer, desktop computer, smartphone, or brainwave data processing system. For example, the depression prediction module 102, or portions thereof, can be implemented as a software application on a computing device, e.g., a server system or stimulus presentation system 106. The wearable device 200 communicates brainwave data received from the sensors 104 to the computing device.

Referring again to FIG. 1, the depression prediction module 102 includes several sub-modules, each of which can be implemented in hardware or software. The depression prediction module 102 includes a stimulus presentation module 108, a stimulus/EEG correlator 110, a depression predictor 112, and a communication module 114. The depression prediction module 102 can be implemented as a software application executed by computing device 118. In some implementations, the sub-modules can be implemented on different computing devices. For example, one or both of the stimulus presentation module 108 and stimulus/EEG correlator 110 can be implemented on the stimulus presentation systems 106 with one or both of the stimulus/EEG correlator 110 and the depression predictor 112 being implemented on a server system (e.g., a cloud server system).

The communication module 114 provides a communication interface for the depression prediction module 102 with the brainwave sensors 104. The communication module 114 can be a wired communication (e.g., USB, Ethernet, fiber optic), wireless communication module (e.g., Bluetooth, ZigBee, WiFi, infrared (IR)). The communication module 114 can serve as an interface with other computing devices, e.g., the stimulus presentation system 106 and user computing devices 130. The communication module 114 can be used to communicate directly or indirectly, e.g., through a network, with the brainwave sensor system 105, the stimulus presentation system 106, user computing devices 130, or a combination thereof.

The stimulus presentation module 108 controls the presentation of stimulus content on the stimulus presentation system 106. The stimulus presentation module 108 can select content to trigger a response by particular brain systems in a patient. For example, the stimulus presentation module 108 can control the presentation of content configured to trigger responses in a dopaminergic system such as an interactive risk/reward game. As another example, the stimulus presentation module 108 can control the presentation of content configured to trigger responses in the amgydala system such as a sequence of emotionally positive, emotionally negative, and emotionally neutral emotional images or video. Moreover, the stimulus presentation module 108 can alternate between appropriate types of content to obtain samples of brain signals from each of one or more particular brain systems. Each brain system provides independent information regarding the patient's diagnosis, resulting in diagnoses that are more and more accurate the more brain systems are probed.

The stimulus presentation module 108 can send data related to the content presented on the stimulus presentation system 106 to the stimulus/EEG correlator 110. For example, the data can include the time the particular content was presented and the type of content. For example, the data can include timestamps indicating a start and stop time of when the content was presented and a label indicating the type of content. The label can indicate which brain system the content targeted. For example, the label can indicate that the presented content targeted a risk/reward system (e.g., the dopaminergic brain system) or an emotion system (e.g., the amygdala). The label can indicate a value of the content, e.g., whether the content was positive, negative, or neutral. For example, the label can indicate whether the content was positive emotional content, negative emotional content, or neutral emotional content. For example, for interactive content, the label can indicate whether the patient made a “winning” or a “losing” selection.

The stimulus/EEG correlator 110 identifies brainwave signals associated with particular brain systems within EEG data from the brainwave sensors 104. For example, the stimulus/EEG correlator 110 receives the EEG data from the brainwave sensors 104 and the content data from the stimulus presentation module 108. The stimulus/EEG correlator 110 can correlate the timing of the content presentation to the patient with the EEG data. That is, the stimulus/EEG correlator 110 can correlate the presentation of the stimulus content with the EEG data to identify brain activity in the EEG data that is responsive to the stimulus. Plot 120 provides an illustrative example. The stimulus/EEG correlator 110 uses the content data to identify EEG data 122 associated with a time period when the stimulus content was presented to the patient, e.g., a stimulus response period (Tₛ). The stimulus/EEG correlator 110 can identify the brainwaves associated with the particular brain system triggered by the content during the stimulus response period (Tₛ). For example, the stimulus/EEG correlator 110 can extract the brainwave data 124 associated with a brain system's response to the stimulus content from the EEG data 122. In some implementations, the stimulus/EEG correlator 110 can tag the EEG data with the start and stop times of the stimulus. In some implementations, the tag can identify they type of content that was presented when the EEG data was measured. In some implementations, the tag can identify what specific image, video, or other type of stimulation was presented (e.g., “the stapler” or “the happy baby.”)

The stimulus/EEG correlator 110 can send the brainwave signals associated with particular types of stimulation and particular brain systems to the depression predictor 112. For example, the stimulus/EEG correlator 110 can send extracted brain wave signals that are associated with one or more brain systems' response to positive images to the depression predictor 112. In some examples, the stimulus/EEG correlator 110 can send tagged brainwave signals where the tags provide information including, but not limited to, an indication of brain system that the brainwaves are associated with, an indication of the type of content presented when the brainwaves were measured, and an indication of where in the brainwave signal the content presentation started.

The depression predictor 112 determines a likelihood that the patient will experience a type of depression in the future, and how severe that depression will be. For example, the depression predictor 112 analyzes brainwave signals associated with one or more brain systems to determine the likelihood that the patient will experience a type of depression, e.g., major depressive disorder or post-partum depression, in the future. In some implementations, the depression predictor 112 analyzes rest state brainwaves, brainwaves associated with the dopaminergic system, brainwaves associated with the amygdala, or a combination thereof.

The depression predictor 112 incorporates a machine learning model to identify patterns in the brainwaves associated with the particular brain systems that are predictive of future depression and depression severity. For example, the depression predictor 112 can include a machine learning model that has been trained to receive model inputs, e.g., detection signal data, and to generate a predicted output, e.g., a prediction of the likelihood that the patient will experience depression in the future, and how severe that depression is likely to be. In some implementations, the machine learning model is a deep learning model that employs multiple layers of models to generate an output for a received input. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a convolutional neural network. In some implementations, the machine learning model is an ensemble of models that may include all or a subset of the architectures described above.

In some implementations, the machine learning model can be a feedforward autoencoder neural network. For example, the machine learning model can be a three-layer autoencoder neural network. The machine learning model may include an input layer, a hidden layer, and an output layer. In some implementations, the neural network has no recurrent connections between layers, or only pruned connections between layers (i.e., is not fully recurrent). Each layer of the neural network may also be fully connected to the next, e.g., there may be no pruning between the layers. The neural network may include an ADAM optimizer for training the network and computing updated layer weights, or may rely on standard gradient descent or other optimization techniques. In some implementations, the neural network may apply a mathematical transformation, e.g., a convolutional transformation or factor analysis, to input data prior to feeding the input data to the network.

In some implementations, the machine learning model can be a supervised model. For example, for each input provided to the model during training, the machine learning model can be instructed as to what the correct output should be. The machine learning model can use batch training, e.g., training on a subset of examples before each adjustment, instead of the entire available set of examples. This may improve the efficiency of training the model and may improve the generalizability of the model. The machine learning model may use folded cross-validation. For example, some fraction (the “fold”) of the data available for training can be left out of training and used in a later testing phase to confirm how well the model generalizes. In some implementations, the machine learning model may be an unsupervised model. For example, the model may adjust itself based on mathematical distances between examples rather than based on feedback on its performance.

A machine learning model can be trained to recognize brainwave patterns from the dopaminergic system, the amygdala, resting state brainwaves, or a combination thereof, that indicate a patient's potential risk of one or more types of depression, and, optionally, how severe that risk is. For example, the machine learning model can correlate identified brainwaves from particular brain system(s) with patterns that are indicative of those leading to a type of depression such as major depressive disorder or post-partum depression. The machine learning model can also correlate identified brainwaves from particular brain system(s) with patterns that are indicative of more or less severe future depression. In some examples, the machine learning model can be trained on hundreds of clinical study data sets based on actual diagnoses of depression. In other examples, the machine learning model can be trained on bootstrapped or non-labelled data. The machine learning model can be trained to identify brainwave signal patterns from relevant brain systems that occur prior to the onset of depression. In some implementations, the machine learning model can refine the ability to predict depression from brainwaves associated brain systems such as those described herein. For example, the machine learning model can continue to be trained on data from actual diagnoses of previously monitored patients that either confirm or correct prior predictions of the model or on additional clinical trial data.

In some examples, the depression predictor 112 can provide a binary output, e.g., a yes or no indication of whether the patient is likely to experience depression. In some examples, the depression predictor 112 provides a risk score indicating a likelihood that the patient will experience depression (e.g., a score from 0-10). In some implementations, the depression predictor can output annotated brainwave graphs. For example, the annotated brainwave graphs can identify particular brainwave patterns that are indicative of future depression. In some examples, the depression predictor 112 can provide a severity score indicating how severe the predicted depression is likely to be.

In some implementations, the depression prediction module 102 sends output data indicating the patient's likelihood of experiencing depression, and, optionally, how severe that depression is likely to be, to a user computing device 130. For example, the depression prediction module 102 can send the output of the depression predictor 112 to a user computing device 130 associated with the patient's doctor.

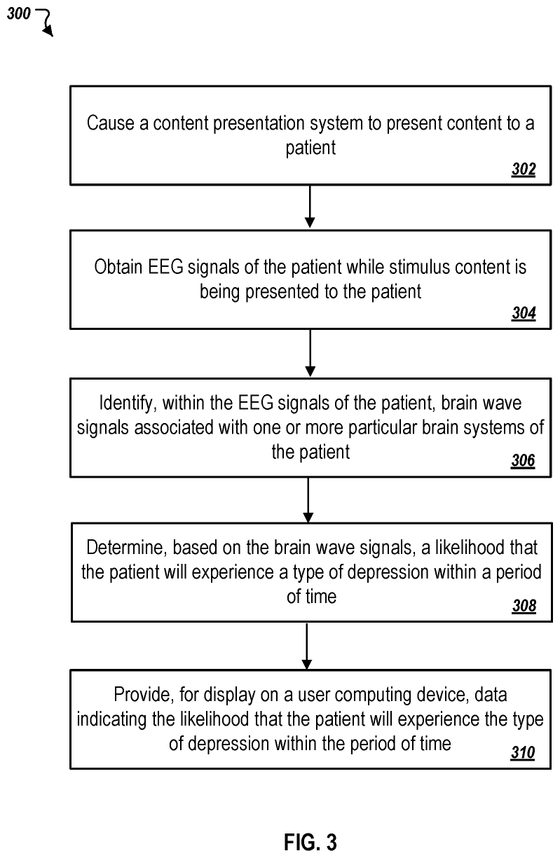

FIG. 3 depicts a flowchart of an example process for using neuroelectric data to predict a patient's likelihood of experiencing depression in the future. In some implementations, the process 300 can be provided as one or more computer-executable programs executed using one or more computing devices. In some examples, the process 300 is executed by a system such as depression prediction module 102 of FIG. 1, or a computing device such as stimulus presentation system 106. In some implementations, all or portions of process 300 can be performed on a local computing device, e.g., a desktop computer, a laptop computer, or a tablet computer. In some implementations, all or portions of process 300 can be performed on a remote computing device, e.g., a server system, e.g., a cloud-based server system.

The system causes a content presentation system to present content to a patient (302). The content can include, but is not limited to, visual content, audio content, and interactive content. For example, the system can control a stimulus presentation system to present content that triggers responses in a particular brain system of a patient. For example, the system can provide risk/reward content to trigger response in a patient's dopaminergic system. The system can provide a sequence emotion eliciting images to trigger responses in a patient's amygdala. The system can provide content instructing the patient to close their eyes and relax, e.g., to obtain resting state brainwaves.

In some examples, the system can alternate between the different types of content. For example, the system can present content to trigger responses in the patient's dopaminergic system first then present content to trigger responses in the patient's amygdala. The system may continue to alternate or to trigger each system multiple times until a diagnosis of high confidence is obtained from the machine learning model.

The system obtains EEG signals of the patient while stimulus content is being presented to the patient (304). For example, the system receives brainwave signals from brainwave sensors worn by the patient while the stimulus content is presented to the patient. In some examples, the system obtains resting state EEG signals when no content is being presented to the patient.

The system identifies, within the EEG signals of the patient, brain wave signals associated with one or more particular brain systems of the patient (306). For example, the system correlates the timing and type of the content presentation with the brainwave signals to identify brainwave signals associated with a particular brain system. For example, the system can correlate the timing of risk/reward content presented to the patient with the brainwave signals to identify brain responses by the patient's dopaminergic system. As another example, the system can correlate the timing of emotion eliciting images presented to the patient with the brainwave signals to identify brain responses by the patient's amygdala and greater emotional processing system.

The system determines, based on the brain wave signals, a likelihood that the patient will experience a type of depression within a period of time, and, optionally, how severe that depression is likely to be (308). For example, the brainwave signals associated with one or more brain systems, and optionally resting state brainwave signals, can be provided as input to a machine learning model. In some implementations, values for parameters from the brainwave signals can, first, be extracted from the time domain brain wave signals and provided as input to the machine learning model. For example, values for a change in signal amplitude over specific time periods can be extracted from the brainwave signals and provides as model input. In some examples, the time periods can correspond to particular time intervals before, concurrent with, and/or after the stimulus content is presented to the patient. In some examples, time periods could also correspond to particular time intervals before, concurrent with, and/or after the patient makes a response to the stimulus consent. For example, values of the brainwave signals within a certain time period (e.g., within 1 second or less, 500 ms or less, 200 ms or less, 100 ms or less) of presentation of a stimulus to the patient can be extracted from the signals and used as input to the machine learning model. More complex features of the brainwave signals can also be extracted and provided as input to the machine learning model. For example, frequency domain,time x frequency domain, regression coefficients, or principal or independent component factors can be provided to the model, instead of or in addition to, raw time domain brainwave signals.

The machine learning model can be, for example, a deep-learning neural network or a “very” deep learning neural network. For example, the machine learning model can be a convolutional neural network. The machine learning model can be a recurrent network. The machine learning model can be an ensemble of all or a subset of these architectures. The machine learning model is trained to predict the likelihood that a patient will experience depression within a period of time in the future, and, optionally, how severe that depression is likely to be, based on detecting patterns indicative of future depression in brainwave signals from one or more brain systems. The model may be trained in a supervised or unsupervised manner. In some examples, the model may be trained in an adversarial manner.

In some implementations, the machine learning model is a supervised model configured to be progressively adaptive to actual patient diagnoses of depression over long periods of time (e.g., two to five years). For example, the machine learning model can receive input indicating actual diagnoses of patients whose brainwaves have been previously analyzed by the model. The model can be tuned, or “learn,” based on the actual diagnoses and whether the actual diagnoses verify or contradict a previous prediction by the model. The machine learning model can be trained to predict the type of depression that a patient may be likely to experience, such as major depressive disorder or post-partum depression, and, optionally, how severe that depression is likely to be.

The machine learning model can be configured to provide a binary output, e.g., a yes or no indication of whether the patient is likely to experience depression. In some examples, the machine learning model is configured to provide a risk score indicating a likelihood that the patient will experience depression (e.g., a score from 0-10). In some examples, the machine learning model is additionally configured to provide a severity score indicating how severe the depression is likely to be (e.g., 1=mild 2=moderate 3=severe). In some implementations, the machine learning model is configured to output annotated brainwave graphs. For example, the annotated brainwave graphs can identify particular brainwave patterns that are indicative of future depression.

The system provides, for display on a user computing device, data indicating the likelihood that the patient will experience the type of depression within the predefined period of time, and, optionally, how severe that depression is likely to be (310). For example, the system can provide the output of the machine learning model to a user computing device associated with the patient's doctor.

Further to the descriptions above, a patient may be provided with controls allowing the user to make an election as to both if and when systems, programs, or features described herein may enable collection of user information. In addition, certain data may be treated in one or more ways before it is stored or used, so that personally identifiable information is removed. For example, a patient's identity may be treated so that no personally identifiable information can be determined for the patient, or a patient's test data and/or diagnosis cannot be identified as being associated with the patient. Thus, the patient may have control over what information is collected about the patient and how that information is used.

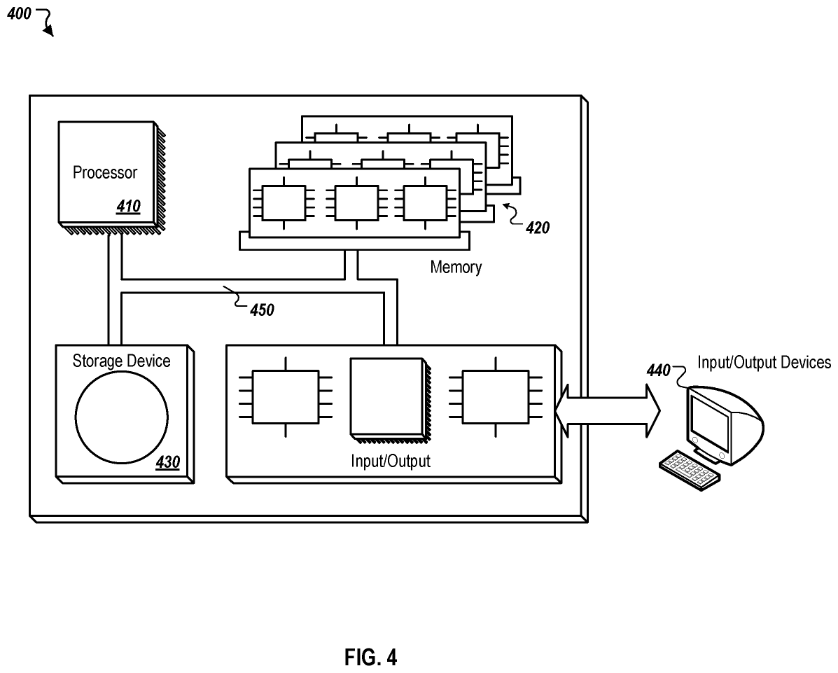

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A depression prediction system, comprising:
one or more processors;
one or more tangible, non-transitory media operably connectable to the one or more processors and storing instructions that, when executed, cause the one or more processors to perform operations comprising:
causing a stimulus presentation system to present first content to a patient, the first content including interactive content configured to test the patient's responses to receiving rewards and taking risks;
obtaining, from a brainwave sensor, electroencephalography (EEG) signals of the patient while the first content is being presented to the patient;
identifying, from within the EEG signals of the patient, first brainwave signals associated with a dopaminergic brain system of the patient, the first brainwave signals representing a response by the patient to the first content;
causing the stimulus presentation system to present second content to the patient, the second content being different from the first content, the second content including a sequence of images representing positive, neutral, and negative emotional stimuli;
obtaining EEG signals of the patient while the second content is being presented to the patient; and
identifying, from within the EEG signals of the patient, second brainwave signals associated with the amygdala and greater emotion processing system of the patient, the second brainwave signals representing a response by the patient to the second content,
determining, based on providing the first brainwave signals and the second brainwave signals as input features to a machine learning model, a likelihood that the patient will experience a type of depression within a period of time; and
providing, for display on a user computing device, data indicating the likelihood that the patient will experience the type of depression within the period of time.

2. The system of claim 1, wherein determining the likelihood that the patient will experience the type of depression within the period of time comprises determining a severity of the type of depression.

3. The system of claim 1, wherein the machine learning model is a convolutional neural network.

4. The system of claim 1, wherein the machine learning model is a supervised machine learning model configured to be adaptive to actual patient diagnoses of depression.

5. The system of claim 1, wherein the machine learning model is trained based on more than one hundred data sets of clinical test data.

6. A computer-implemented depression prediction method executed by one or more processors and comprising:
causing, by the one or more processors, a stimulus presentation system to present first content to a patient;
obtaining, by the one or more processors and from a brainwave sensor, electroencephalography (EEG) signals of the patient while the first content is being presented to the patient;
identifying, by the one or more processors and from within the EEG signals of the patient, first brainwave signals associated with a first brain system of the patient, the first brainwave signals representing a response by the patient to the first content;
determining, based on providing the first brainwave signals as input features to a machine learning model, a likelihood that the patient will experience a type of depression within a period of time; and
providing, for display on a user computing device, data indicating the likelihood that the patient will experience the type of depression within the period of time.

7. The method of claim 6, wherein the first content is selected to trigger a response by a particular brain system of the patient.

8. The method of claim 6, wherein the first brain system is a dopaminergic system or amygdala emotional system.

9. The method of claim 6, further comprising:
causing the stimulus presentation system to present second content to the patient, the second content being different from the first content;
obtaining EEG signals of the patient while the second content is being presented to the patient; and
identifying, from within the EEG signals of the patient, second brainwave signals associated with a second brain system of the patient, the second brainwave signals representing a response by the patient to the second content,
wherein determining the likelihood that the patient will experience the type of depression within the period of time comprises determining, based on providing the first brainwave signals and the second brainwave signals as input features to the machine learning model, the likelihood that the patient will experience the type of depression within the period of time.

10. The method of claim 9, wherein the first brain system is a reward system and the second brain system is an emotion system.

11. The method of claim 9, further comprising:
obtaining EEG signals of the patient while no content is presented to the patient; and
identifying, from within the EEG signals of the patient, third brainwave signals associated with a resting state of the patient,
wherein determining the likelihood that the patient will experience the type of depression within the period of time comprises determining the likelihood that the patient will experience the type of depression within the period of time based on the first brainwave signals, the second brainwave signals, and the third brainwave signals.

12. The method of claim 6, wherein determining the likelihood that the patient will experience the type of depression within the period of time comprises determining a severity of the type of depression.

13. The method of claim 6, wherein the machine learning model is a convolutional neural network.

14. The method of claim 6, wherein the machine learning model is a supervised machine learning model configured to be adaptive to actual patient diagnoses of depression.

15. The method of claim 6, wherein the machine learning model is trained based on more than one hundred data sets of clinical test data.

16. The method of claim 6, wherein the type of depression includes major depressive disorder or post-partum depression.

17. The method of claim 6, wherein the first content includes interactive content configured to test the patient's responses to receiving rewards and taking risks.

18. The method of claim 6, wherein the first content includes a sequence of images representing positive, neutral, and negative emotional stimuli.

19. A non-transitory computer readable storage medium storing instructions that, when executed by at least one processor, cause the at least one processor to perform operations comprising:
causing a stimulus presentation system to present first content to a patient;
obtaining, by the one or more processors and from a brainwave sensor, electroencephalography (EEG) signals of the patient while the first content is being presented to the patient;
identifying, from within the EEG signals of the patient, first brainwave signals associated with a first brain system of the patient, the first brainwave signals representing a response by the patient to the first content;
determining, based on providing the first brainwave signals as input features to a machine learning model, a likelihood that the patient will experience a type of depression within a period of time; and
providing, for display on a user computing device, data indicating the likelihood that the patient will experience the type of depression within the period of time.

20. The medium of claim 19, wherein determining the likelihood that the patient will experience the type of depression within the period of time comprises determining a severity of the type of depression.
